(19) United States
(12) Patent Application Publication
(10) Pub. No.: US 2023/0287340 A1
(43) Pub. Date: Sep. 14, 2023

(54) METHODS FOR SPERMATOGONIAL CULTURE

(71) Applicant: Washington State University, Pullman, WA (US)

(72) Inventors: Jon Michael Oatley, Pullman, WA (US); Melissa Joan Oatley, Pullman, WA (US)

(73) Assignee: Washington State University, Pullman, WA (US)

(21) Appl. No.: 18/006,009

(22) PCT Filed: Jul. 27, 2021
(86) PCT No.: PCT/US2021/070967

§ 371 Date: Jan. 19, 2023

Related U.S. Application Data
(60) Provisional application No. 63/057,245, filed on Jul. 27, 2020.

(51) Int. Cl. C12N 5/076; A61K 35/52

(52) U.S. Cl. CPC C12N 5/061; A61K 35/52; C12N 2501/13; C12N 2501/115; C12N 2501/21; C12N 2501/22; C12N 2501/15; C12N 2502/28

(57) ABSTRACT

This disclosure provides improved method, kits, and systems and for isolating and enriching spermatogonial stem cells (SSCs) from livestock animal testicular tissue. In one aspect, the disclosure provides a method for enriching SSCs from a population of testis-derived cells containing at least one SSC, where the method comprises contacting the population of testis-derived cells to a culture media comprising, or that is preconditioned with, endothelial feeder cells, and maintaining culture conditions suitable for SSC cell maintenance and enrichment. In some embodiments, the media and culture conditions comprise one or more growth factors selected from GDNF, FGF2, SDF-1a, CSF-1, FDGF, NGF, and TGF-β, in any combination. In exemplary embodiments, the SSCs are porcine or bovine SSCs.

### CROSS-REFERENCE TO RELATED APPLICATION

This application claims the benefit of Provisional Application No. 63/057,245, filed Jul. 27, 2020, the disclosure of which is incorporated herein by reference in its entirety

### STATEMENT OF GOVERNMENT LICENSE RIGHTS

This invention was made with Government support under grant numbers 2008-35203-31209 and 2013-67015-20996 awarded by United States Department of Agriculture through the National Institute of Food & Agriculture. The Government has certain rights in the invention.

### BACKGROUND

Stem cells are undifferentiated cells that possess two hallmark properties: self-renewal and the ability to differentiate into one or more different cell lineages. The process of self-renewal involves the self-replication of a stem cell to allow for propagation and expansion, wherein the stem cell remains in an undifferentiated state. Progenitor cells are also undifferentiated cells that have the ability to differentiate into one or more cell lineages but have limited or no ability to self-renew. When maintained in culture, undifferentiated cells, such as stem or progenitor cells, can undergo spontaneous differentiation, thereby losing the desired, undifferentiated cell phenotype. Thus, culture methods that minimize spontaneous differentiation in order to maintain the undifferentiated stem or progenitor cell state are needed.

Keeping undifferentiated cells in an undifferentiated state is critical to their use, e.g., in industry and medicine, because a major scientific and therapeutic usefulness of these cells lies in their ability to expand into homogenous populations that can further proliferate or differentiate into mature cells as needed, e.g., for scientific study or to repair damage to cells or tissues of a patient. Once they have spontaneously differentiated in cell culture, the cells are less proliferative and less able to differentiate into different types of cells as needed. A homogenous culture of undifferentiated stem cells is therefore a highly sought after but unrealized goal of research scientists and industry.

The ability to conditionally induce the development of stem cell lines through the process of spermatogenesis in vitro for the production of gametes would provide a long-sought-after technology for biomedical research and animal breeding, particularly if such protocols could be established for a variety of species. To date, most success has been achieved in rats and mice only, leaving larger mammals such as cows, pigs, horses, and the like, without such advances.

Extant procedures to isolate, maintain, and expand testis cells that retain the ability to undergo spermatogenesis in the testes of a recipient animal remains complex and largely unsuccessful. For example, media for long-term proliferation of rodent spermatogonial stem cells in vitro are relatively complex, expensive, time-consuming to prepare, plus are most effective when applied in combination with feeder layers of fibroblasts.

Despite the advances in the art of spermatogonial stem cell culture, a need exists for facile and effective methods of culturing and expanding spermatogonial stem cells, particularly for larger mammals. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of enriching spermatogonial stem cells (SSCs) from a population of testis-derived cells containing at least one SSC, wherein the population of cells is derived from the testis of a livestock animal. The method comprises: contacting said population of testis-derived cells to a culture media comprising endothelial feeder cells, or parts thereof, and maintaining culture conditions suitable for SSC cell maintenance and enrichment.

In one embodiment, the endothelial feeder cells are maintained in a layer in or on a gelatin coated culture surface. In one embodiment, the feeder cells are embryonic endothelial cells. In one embodiment, the feeder cells are yolk sac-derived endothelial cells. In one embodiment, the feeder cells are characterized by expression of vascular addressin. In one embodiment, the feeder cells are characterized by expression of cytoplasmic tyrosine kinase encoded by a fes (fps/fes) oncogene or homologs thereof. In one embodiment, the feeder cells are murine C166 cells. In one embodiment, the feeder cells are pre-treated with a mitosis inactivating agent, such as Mitomycin-C.

In come embodiments, the culture media further comprises one or more (e.g., two, three, four, five, six or all) growth factors selected from glial cell-derived neurotrophic factor (GDNF), fibroblast growth factor 2 (FGF2), stromal cell-derived factor 1 (SDF-1a), colony stimulating factor 1 (CSF-1), fibroblast-derived growth factor (FDGF), nerve growth factor (NGF), and transforming growth factor beta (TGF-β), in any combination. In one embodiment, the culture media comprises GDNF, FGF2, SDF-1a, and CSF-1. In one embodiment, the culture media further comprises DGF, NGF, and TGF-β.

In one embodiment, the method further comprises isolating enriched SSCs from the culture media.

In one embodiment, the SSCs are porcine SSCs. In a specific embodiment, the SSCs are porcine SSCs, wherein the endothelial feeder cells comprise murine C166 cells, and wherein the culture media further comprises GDNF, FGF2, SDF-1a, and CSF-1. In one embodiment, the SSCs are bovine SSCs. In one embodiment, the SSCs are bovine SSCs, wherein the endothelial feeder cells comprise murine C166 cells, and wherein the culture media further comprises GDNF, FGF2, SDF-1a, CSF-1, DGF, NGF, and TGF-β.

In another aspect, the disclosure provides a method of enriching spermatogonial stem cells (SSCs) from a population of testis-derived cells containing at least one SSC, wherein the population of cells is derived from the testis of a livestock animal. The method comprises: contacting the population of testis-derived cells to a culture media that has been preconditioned with a feeder cell line of endothelial cells, or parts thereof, and maintaining culture conditions suitable for SSC cell maintenance and enrichment.

In one embodiment, the culture media is preconditioned by contacting the media with the endothelial feeder cells which are maintained in a layer in or on a gelatin coated culture surface. In one embodiment, the feeder cells are embryonic endothelial cells. In one embodiment, the feeder cells are yolk sac-derived endothelial cells. In one embodiment, the feeder cells are characterized by expression of vascular addressin. In one embodiment, the feeder cells are characterized by expression of cytoplasmic tyrosine kinase encoded by a fes (fps/fes) oncogene or homologs thereof. In one embodiment, the feeder cells are murine C166 cells. In one embodiment, the feeder cells are treated with a mitosis inactivating agent, such as Mitomycin-C, prior to or during contact with the culture media.

In one embodiment, the culture media further comprises one or more (e.g., two, three, four, five, six or all) growth factors selected from GDNF, FGF2, SDF-1a, CSF-1, FDGF, NGF, and TGF-β, in any combination. In one embodiment, the culture media comprises GDNF, FGF2, SDF-1a, and CSF-1. In one embodiment, the culture media further comprises FDGF, NGF, and TGF-β.

In one embodiment, the method further comprises preconditioning the culture media with the feeder cell line of endothelial cells, followed by removing the feeder cell line of endothelial cells or substantially all of the feeder cell line of endothelial cells from the preconditioned culture media prior to contacting preconditioned culture media to the population of testis-derived cells. In one embodiment, the method further comprises isolating enriched SSCs from the preconditioned culture media.

In one embodiment, the SSCs are porcine SSCs. In a specific embodiment, the SSCs are porcine SSCs, wherein the endothelial feeder cells comprise murine C166 cells, and wherein the culture media further comprises GDNF, FGF2, SDF-1a, and CSF-1. In one embodiment, the SSCs are bovine SSCs. In one embodiment, the SSCs are bovine SSCs, wherein the endothelial feeder cells comprise murine C166 cells, and wherein the culture media further comprises GDNF, FGF2, SDF-1a, CSF-1, DGF, NGF, and TGF-β.

In another aspect, the disclosure provides a population of enriched SSC produced by the method as described herein.

In another aspect, the disclosure provides a method of generating at least one livestock animal progeny. The method comprises:
a) administering a population of enriched SSCs as described herein to a testis of a recipient male livestock animal;
b) allowing said enriched SSCs to generate a colony of spermatogenesis in said recipient male livestock animal; and
c) mating said recipient male livestock animal with a female male livestock mammal of the same species as said recipient male livestock animal.

In one embodiment, the population of enriched SSCs is administered to the rete testis of said recipient male livestock animal. In one embodiment, the population of enriched SSCs is administered to the lumen of a seminiferous tubule of said recipient male livestock animal. In one embodiment, the SSCs are porcine cells and the recipient livestock animal and the female livestock animal are porcine animals. In one embodiment, the SSCs are bovine cells and the recipient male livestock animal and the female livestock animal are bovine animals. In one embodiment, the recipient male livestock animal is genetically modified to reduce or eliminate NANOS2 function.

In another aspect, the disclosure provides a kit for maintaining spermatogonial stem cells (SSCs) in a feeder system, the kit comprises:
a) a culture system comprising a plurality of endothelial feeder cells and a medium suitable for SSC cell growth;
b) an applicator, and
c) instructional material, wherein said instructional material comprises instructions for the use of said kit to maintain the SSCs in the culture system.

In one embodiment, the kit further comprises a container with one or more culture chambers configured to receive the medium containing the endothelial feeder cells and the SSCs

In one embodiment, the container comprises a gelatin coated culture surface. In one embodiment, the feeder cells are embryonic endothelial cells. In one embodiment, the feeder cells are yolk sac-derived endothelial cells. In one embodiment, the feeder cells are characterized by expression of vascular addressin and/or cytoplasmic tyrosine kinase encoded by a fes (fps/fes) oncogene or homologs thereof. In one embodiment, the feeder cells are murine C166 cells. In one embodiment, the feeder cells are pretreated with a mitosis inactivating agent, such as Mitomycin-C. In one embodiment, the kit further comprises a mitotic inactivating agent, such as Mitomycin-C. In one embodiment, the culture media comprises one or more growth factors selected from GDNF, FGF2, SDF-1a, CSF-1, FDGF, NGF, and TGF-β, in any combination.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a representative image of primary culture of porcine spermatogonia taken during long-term culture using the disclosed strategy. The image was taken after 30 days of in vitro culture.

FIG. 2 graphically illustrates the porcine quantification of spermatogonial number over a 2-month period of in vitro maintenance. Data are for 5 different cultures, all maintained in the same culture conditions.

FIGS. 3A to 3C are micrographs illustrating primary cultures of porcine spermatogonia. FIG. 3A illustrates brightfield image of live spermatogonia cells in culture. FIG. 3B and FIG. 3C illustrate immunocytochemical staining of spermatogonial cells using DAPI (for DNA) and the molecular marker VASA or IgG for negative control in brightfield. These images establish that the primary cultures are germ cells.

FIGS. 4A and 4B illustrate flow cytometric analysis of primary porcine spermatogonial cultures for the stem cell marker SOX2 and negative control (IgG). These scatter plots establish that the cultures have molecular characteristics of stem cells.

FIG. 5 is a representative image of spermatozoa derived from primary cultures of porcine spermatogonia transplanted into the testes of a NANOS2 knockout surrogate sire boar. This establishes that the porcine primary cultures possess spermatogenic stem cell capacity even after prolonged in vitro culture and expansion.

FIG. 6 is a representative image of primary culture of bovine spermatogonia taken during long-term culture using the disclosed strategy. The image was taken after 30 days of in vitro culture with C166 endothelial cell feeder cells.

FIG. 7 graphically illustrates the growth curve for primary cultures of bovine spermatogonia maintained in vitro over for an 80-day period with C166 endothelial cell feeder cells. Data are mean±SEM for n=3 biological replicate cultures.

### DETAILED DESCRIPTION

The present disclosure is based on the inventors' development of an improved methodology for isolating and expanding spermatogonial stem cells (SCCs) from testicular tissue of livestock animals (e.g., porcine or bovine testicular tissue) using endothelial feeder cells to create a unique culture environment. As discussed in more detail below, the disclosed methods resulted in unexpected and remarkable, e.g., exponential, expansion in an ex vivo culture at a rate that has not heretofore been achieved, especially in livestock animals. The expanded SCCs were demonstrated to have normal morphology and were successfully implanted in recipient surrogate males leading to spermatogenesis of apparently normal sperm. Accordingly, the disclosed methods and reagents are highly useful for implantation in recipient animals, e.g., male porcine or bovine animals, to promote controlled animal breeding strategies.

Co-Culture Methods

In accordance with the foregoing, in one aspect the disclosure provides a method of enriching spermatogonial stem cells (SSCs) from a population of testis-derived cells containing at least one SSC. The population of testis-derived cells is derived from the testis of a livestock animal. In one embodiment, the method comprises contacting said population of testis-derived cells to a culture media comprising endothelial feeder cells, or parts thereof, and maintaining culture conditions suitable for SSC cell maintenance and enrichment. In an alternative embodiment, the method comprises contacting said population of testis-derived cells to a culture media that has been preconditioned endothelial feeder cells, but from which the endothelial cells have been substantially eliminated.

As used herein, the phrase “spermatogonial stem cells” or “SSCs” denotes stem cells either isolated from the testis, created from induced pluripotent stem cells, from embryonic stem cells or any other method to obtain such cells. For example, mammalian induced pluripotent stem cells created from skin cells have been used to create germ cells. See, Easley CA 4th et al, Cell Rep. 2012 Sep. 27; 2(3):440-6, “Direct differentiation of human pluripotent stem cells into haploid spermatogenic cells”. Spermatogonial stem cells are incapable of fertilizing an egg cell but, under the appropriate conditions, can give rise to cells that develop into sperm through the process of spermatogenesis and so produce viable offspring. Isolated spermatogonial stem cells can be cultured for a prolonged time period without losing their properties and can efficiently repopulate the testes of suitable recipient male animals described, for instance, in Oatley, Jon M., and Ralph L. Brinster. “Spermatogonial stem cells.” Methods in Enzymology 419 (2006): 259-282.

The disclosure encompasses SSCs derived from the testis of livestock animals. The term “livestock animal” includes animals traditionally raised in livestock farming, such as bovine animals (e.g., beef cattle, dairy cattle, buffalo, bison, etc.), porcine animals (e.g., pigs), sheep, goats, horses, mules, asses, and camels. In some embodiments, the term also includes birds raised commercially for meat or eggs (i.e., chickens, turkeys, ducks, geese, guinea fowl, and squabs). The term “livestock animal” does not include does not include rodents (e.g., rats or mice) or lagomorphs (e.g., rabbits). In a specific embodiment, the SSCs are porcine cells (e.g., from the genus Sus, such as S. scrofa). In another specific embodiment, the SSCs are bovine cells (e.g., from the genus Bos, such as B. taurus). In another specific embodiment, the SSCs are goat cells (e.g., from the genus Capra, such as Capra aegagrus). Regardless of the species of donor animal, the cells can be derived from, e.g., one of the following: wild type adult testis, pup testis, neonate testis, induced pluripotent cells, embryonic stem cells, and/or cryptorchid adult testis. As used herein, the term “enriching” refers to the process by which the concentration, number, or activity of something (e.g., SSCs) is increased from a prior state. For example, a population of 100 spermatogonial stem cells is considered to be “enriched” in spermatogonial stem cells if the population previously contained only 50 spermatogonial stem cells. Similarly, a population of 100 spermatogonial stem cells is also considered to be “enriched” in spermatogonial stem cells if the population previously contained 99 spermatogonial stem cells. Likewise, a population of 100 spermatogonial stem cells is also considered to be “enriched” in spermatogonial stem cells even if the population previously contained zero spermatogonial stem cells.

As the term is used herein, “population” refers to two or more cells.

A cell or population of cells is “testis-derived”, as the term is used herein, if the cell or cell population is obtained or derived from a testis. By way of a non-limiting example, testis-derived cells include a spermatogonial stem cell, a somatic cell, and a germ cell. Typically, the cell or cell population is surgically removed from testis tissue. The cell or cell population can be further processed and selected or isolated from the dissected tissue. An exemplary, non-limiting protocol for obtaining or deriving a cell population from testis tissue is provided in the examples below.

As indicated above, in some embodiments the culture media comprises endothelial feeder cells. Feeder cells are a population of cells that are used to influence the culture environment in a manner that is beneficial for the maintenance and/or expansion of a cell population of interest (e.g., SSCs). The endothelial feeder cells can be endothelial cells that are transformed (i.e., immortalized) or endothelial cells that are not transformed but rather obtained from a source organism or tissue. In some embodiments, the endothelial feeder cells are embryonic endothelial cells. In some embodiments, the endothelial feeder cells are derived from the yolk sac. For example, the endothelial feeder cells can be primary yolk sac derived cells without immortalization. In alternative embodiments, the feeder cells are immortalized yolk save derived cells. The endothelial feeder cells can be derived from any organism, such as human, rodent (e.g., mouse, rat), rabbit, goat, cow, pig, and the like, regardless of the identity or source of the SSCs. For example, the SSCs can be porcine or bovine, whereas the endothelial feeder cells can be of murine origin. In other embodiments, the feeder cells are derived from the same species as the SSCs cultured in the method.

In some embodiments, the endothelial feeder cells are characterized by expression of identifiable markers characteristic of certain endothelial cell lines. For example, in some embodiments, the endothelial feeder cells are characterized by expression of vascular addressin or homologs thereof. In some embodiments, the feeder cells are characterized by expression of cytoplasmic tyrosine kinase encoded by a fes (fps/fes) oncogene or homologs thereof. In some embodiments, the feeder cells are engineered to contain and express the human fes gene to produce the fps/fes enzyme. Other potential non-limiting characteristics of the feeder cells include expression of vascular cell-adhesion molecule 1 (CD106, VCAM-1) or a homolog thereof, acetylated low density lipoprotein (LDP) or a homolog thereof, angiotensin converting enzyme (ACE) or a homolog thereof. In a specific embodiment, the feeder cells are murine C166 cells, which are available from ATCC, cat #CRL-2581. In a further embodiment, the C166 cells are transformed to express a fes oncogene, such as the human fes oncogene.

The endothelial feeder cells can be maintained in a layer in or on a gelatin coated culture surface, such as in a plastic or glass culture plate or well. The endothelial feeder cells can be pre-treated in a manner that renders them unable to divide or proliferate. For example, the feeder cells can be exposed with a sufficient dose of radiation to render them incapable of undergoing mitosis, but leaving them with an active and stable metabolism. Alternatively, the feeder cells can be exposed to a mitosis inactivating agent. An illustrative, non-limiting example of a mitosis inactivating agent is Mitomycin-C. Other mitosis inactivating agents that result in the cessation mitosis in a cell while maintaining an active and stable metabolism are known and encompassed by this disclosure.

The culture media containing, inter alia, the endothelial feeder cells can be a “defined culture media”, which refers to the components of the media comprising or consisting of known ingredients at known proportions or quantities. The base culture media can be, e.g., DMEM. The base culture media can further comprise various components familiar in the art to facilitate maintenance or expansion of the SSCs. Illustrative, non-limiting examples of media components include BME, Na₂SeO₃, transferring, putrescine, vitamins, NEAA, Hepes, glutamine, antibiotics (e.g., penicillin or streptomycin), serum (e.g., BSA) or serum substitute (e.g., StemPro). Exemplary formulations for culture media encompassed by this disclosure are described in more detail in Examples 1 and 3.

In some embodiments, the culture media further comprises one or more growth factors to facilitate SSC maintenance and enrichment. Exemplary growth factors include at least one of the growth factors selected from glial cell-derived neurotrophic factor (GDNF), fibroblast growth factor 2 (FGF2), stromal cell-derived factor 1 (SDF-1a), colony stimulating factor 1 (CSF-1), fibroblast-derived growth factor (FDGF), nerve growth factor (NGF), and transforming growth factor beta (TGF-β). These growth factors from different origins, e.g., human, etc., can be commercially obtained (see reagents section in Example 1). In embodiments of multiple growth factors, e.g., two, three, four, five, six, or all of the growth factors selected from GDNF, FGF2, SDF-1a, CSF-1, DGF, NGF, and TGF-β, the growth factors can be included in any combination. Exemplary combinations include at least GDNF and FGF2, at least GDNF and SDF-1a, at least GDNF and CSF-1, at least GDNF and DGF, at least GDNF and NGF, at least GDNF and TGF-β, at least FGF2 and SDF-1a, at least FGF2 and CSF-1, at least FGF2 and DGF, at least FGF2 and NGF, at least FGF2 and TGF-β, at least SDF-1a and CSF-1, at least SDF-1a and DGF, at least SDF-1a and NGF, at least SDF-1a and TGF-β, at least CSF-1 and DGF, at least CSF-1 and NGF, at least CSF-1 and TGF-β, at least DGF and NGF, at least DGF and TGF-β, and at least NGF and TGF-β from the aforementioned list. In some embodiments, the culture media comprises at least the growth factors GDNF, FGF2, SDF-1a, and CSF-1. Such an embodiment can be particularly applicable to culture and expansion of porcine SSCs, as described in Examples 1 and 2. In some embodiments, the culture media comprises the growth factors GDNF, FGF2, SDF-1a, CSF-1, DGF, NGF, and TGF-β. Such an embodiment can be especially applicable to culture and expansion of bovine SSCs as described in Example 3.

The aforementioned growth factors, whether present individually or in any combination, can be applied at any appropriate concentration to facilitate maintenance and/or enrichment of the SSCs. Illustrative, non-limiting concentrations of GDNF, whether present alone or in combination with other growth factors, include from about 5 ng/ml to about 40 ng/ml, such as from about 10 ng/ml to about 30 ng/ml, and from about 15 ng/ml to about 25 ng/ml, including about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 ng/ml. Illustrative, non-limiting concentrations of FGF2, whether present alone or in combination with other growth factors, include from about 0.1 ng/ml to about 4 ng/ml, such as from about 0.5 ng/ml to about 3 ng/ml, from about 1 ng/ml to about 2.5 ng/ml, and from about 1.5 ng/ml to about 2 ng/ml, including about 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0 ng/ml. Illustrative, non-limiting concentrations of CSF-1, whether present alone or in combination with other growth factors, include from about 1 ng/ml to about 20 ng/ml, such as from about 5 ng/ml to about 15 ng/ml, and from about 7 ng/ml to about 12 ng/ml, including about 1, 2, 3, 4, 5, 6, 7, 8, 9, 10, 11, 12, 13, 14, 15, 16, 17, 18, 19, and 20 ng/ml. Illustrative, non-limiting concentrations of SDF-1a, whether present alone or in combination with other growth factors, include from about 1 ng/ml to about 20 ng/ml, such as from about 5 ng/ml to about 15 ng/ml, and from about 7 ng/ml to about 12 ng/ml, including about 1, 2, 3, 4, 5, 6, 7, 8, 9, 10, 11, 12, 13, 14, 15, 16, 17, 18, 19, and 20 ng/ml. Illustrative, non-limiting concentrations of PDGF, whether present alone or in combination with other growth factors, include from about 1 ng/ml to about 20 ng/ml, such as from about 5 ng/ml to about 15 ng/ml, and from about 7 ng/ml to about 12 ng/ml, including about 1, 2, 3, 4, 5, 6, 7, 8, 9, 10, 11, 12, 13, 14, 15, 16, 17, 18, 19, and 20 ng/ml. Illustrative, non-limiting concentrations of NGF, whether present alone or in combination with other growth factors, include from about 1 ng/ml to about 20 ng/ml, such as from about 5 ng/ml to about 15 ng/ml, and from about 7 ng/ml to about 12 ng/ml, including about 1, 2, 3, 4, 5, 6, 7, 8, 9, 10, 11, 12, 13, 14, 15, 16, 17, 18, 19, and 20 ng/ml. Illustrative, non-limiting concentrations of TGF-β, whether present alone or in combination with other growth factors, include from about 1 ng/ml to about 20 ng/ml, such as from about 5 ng/ml to about 15 ng/ml, and from about 7 ng/ml to about 12 ng/ml, including about 1, 2, 3, 4, 5, 6, 7, 8, 9, 10, 11, 12, 13, 14, 15, 16, 17, 18, 19, and 20 ng/ml. It will be appreciated that the described concentrations can be applied in any combination for the different growth factors present in, e.g., the combinations described above. To illustrate, in an exemplary combination, the culture media comprises about 18-22 ng/ml (e.g., 20 ng/ml) GDNF, about 0.5-2.5 ng/ml (e.g., 1 or 2 ng/ml) FGF2, about 8-12 ng/ml (e.g., about 10 ng/ml) CSF-1, and about 8-12 ng/ml (e.g., about 10 ng/ml) SDF-1a. This embodiment can be particularly useful for maintaining and expanding porcine SSCs. In another embodiment, in addition to the immediately forementioned combinations and concentrations, the culture media further comprises about 8-12 ng/ml (e.g., about 10 ng/ml) PDGF, about 8-12 ng/ml (e.g., about 10 ng/ml) NGF, and about 8-12 ng/ml (e.g., about 10 ng/ml) TGF-β. This embodiment can be particularly useful for maintaining and expanding bovine SSCs.

The method comprises maintaining culture conditions suitable for SSC cell maintenance and enrichment. Such culture conditions include provision fresh culture media, including defined media with the exemplary components (e.g., growth factors) described above. Additionally, the culture conditions include providing appropriate temperature and oxygen tension. Appropriate culture temperatures include between about 34° C. and 38° C., inclusive, such as 34° C., 35° C., 36° C., 37° C., and 38° C. Appropriate O₂ tension levels include between about 7% O₂ and 13% O₂, inclusive, such as 7% O₂, 8% O₂, 9% O₂, 10% O₂, 11% O₂, 12% O₂, 13% O₂, 14% O₂, and 15% O₂. In a specific embodiment, the culture conditions are maintained at about 35° C. and 10% O₂.

The method produces a population of enriched SSCs, as demonstrated in the Examples below. In some embodiments, the method further comprises isolating the enriched SSCs from the culture media. The enriched SSCs are isolated or substantially isolated from the feeder cells for further application, e.g., in livestock animal breeding programs. As used herein, “substantially isolated from” refers to the removal of population of first substances (e.g., expanded SSCs) from the proximity of a population of second substances (e.g., feeder cells). The population of first substances is not necessarily devoid of the second substance, and the population of second substances is not necessarily devoid of the first substance. However, a population of first substances that is “substantially isolated from” a population of second substances has a measurably lower content of second substances as compared to the non-separated mixture of first and second substances.

While the above embodiments of the method are generally described in the context of enriching spermatogonial stem cells (SSCs) in a co-culture with the endothelial feeder cells, the disclosure also encompasses “feeder-free” embodiments. In feeder-free embodiments, the population of testis-derived cells (i.e., from the testis of a livestock animal) is contacted to a culture media that has been preconditioned by endothelial feeder cells or parts thereof. In this embodiment, the culture media if first contacted with the feeder cells for a time sufficient for the feeder cells to confer beneficial qualities on the media and then is then removed from the feeder cell population. The preconditioned media can be used immediately thereafter or stored for a time period prior to contacting with the testis-derived cells.

The preconditioned media can be free or substantially free of the feeder cells or cell parts. As used herein, the term “substantially free” encompasses embodiments where a minor component of feed cells or cell parts remain with the preconditioned medium but are sufficiently minimized as to have insignificant impact on the continued functionality of the preconditioned media. For example, any remaining feeder cells will no longer exert one or more of a physical, biological or chemical effect on the culture medium. For example, the preconditioned media cell can be substantially free of feeder cells if at least 75% (e.g., 80%, 85%, 90%, 95%, 98%, etc.) of the feeder cells are removed from the culture media. The preconditioned media can be separated from the feeder cells by known techniques, including allowing cells to fall to the bottom of the culture vessel and aspirating or pouring off the preconditioned medium. This process can be facilitated by centrifugation. The medium can also be subjected filtration to remove additional cells parts. In some embodiments, the preconditioned medium is created by contacting the media with the feeder cells for a time sufficient for the cells to detectably alter the composition of the media. The preconditioned media can thereafter be stored appropriately and/or contacted with the population of testis-derived cells.

In another aspect, the disclosure provides an enriched SSC or population of enriched SSCs produced by the method disclosed herein. As described below, the methods produce cultured SSCs that can reproduce and expand at a high rate while retaining the pluripotency of germ cells. The cell or population of cells can be maintained in cultured state with an active metabolism, or preserved (e.g., frozen) while retaining the capacity to return to an active metabolic state. An SSC can be identified as being “maintained” in culture (e.g., in the culture system disclosed herein) by assessing the activity of an SSC at various time points in the culture medium and comparing the activity with the activity of the SSCs at the start of the culture period. As will be understood by the skilled artisan, little or no loss of activity is an indication that SSCs have been maintained in culture.

In another aspect, the disclosure provides a method of transplanting one or more SSCs to a recipient livestock animal. The method can be incorporated into a method of generating at least one livestock animal progeny. The method comprises:
a) administering a population enriched SSCs, produced as described herein, to a testis of a male recipient livestock animal;
b) allowing said enriched SSCs to generate a colony of spermatogenesis in said recipient male livestock animal; and
c) mating said male recipient livestock animal with a female livestock animal of the same species as said recipient male livestock animal.

The male recipient livestock animal can be any livestock animal as described above. In some embodiments, the livestock animal is selected from bovine animals (e.g., beef cattle, dairy cattle, buffalo, bison, etc.), porcine animals (e.g., pigs), sheep, goats, horses, mules, asses, and camels. In some embodiments, the term also includes birds raised commercially for meat or eggs (i.e., chickens, turkeys, ducks, geese, guinea fowl, and squabs). The term “livestock animal” does not include does not include rodents (e.g., rats or mice) or lagomorphs. In a specific embodiment, the SSCs are porcine cells and the male recipient livestock animal and the female livestock animal are pigs. In another embodiment, the SSCs are bovine cells and the recipient male livestock animal and the female livestock animal are bovine.

Transplantation methods are generally known in the art and are be discussed in extensive detail herein. For general cell transplantation methods involving the testis, see Kanatsu-Shinohara et al. (PNAS, 99:1383-1388 (2002)), Brinster (I) (U.S. Pat. No. 6,215,039), and Brinster (II) (U.S. Pat. No. 5,858,354), all of which are hereby incorporated by reference herein in their entirety. Brinster (I) and (II) demonstrate, in part, that SSCs transplanted from a donor to an immunologically tolerant mouse or other compatible recipient will replicate and be maintained in the recipient. WO 2016/011029, incorporated herein by reference in its entirety, describes transplantation methods implanted in, e.g., porcine and bovine, surrogate animals that have been genetically modified to disrupt NANOS2 gene function. The knockout of this specific target results in ablation of germ cell production, but where the resulting testis tissue retains the functional somatic cells that support spermatogenesis from implanted spermatogonia. Such animals are encompassed by the present disclosure.

In an embodiment, one or more SSCs are introduced into the tubules of a testis, e.g., the lumen of a seminiferous tubule. For example, a recipient male livestock animal can be anesthetized and the testis (or testes) surgically exposed. In one embodiment, using micromanipulation methods, a thin glass needle is introduced into exposed tubules, one after another, and each tubule is injected with a solution containing the primitive cells being used to colonize the tubule. In another embodiment, one or more SSCs can also be introduced by injecting them into other parts of the tubular system, e.g., the lumen of the rete testes. As will be understood by the skilled artisan, injection methods are available that minimize the number of injection sites and increase the efficiency of injection of SSCs into a recipient male.

A cell suspension of one or more SSCs for injection can comprise an injection medium and at least one SSC at a suitable concentration. By way of a non-limiting example, the injection medium can comprise one or more of NaCl, Na₂HPO₄, KCl, KH₂PO₄, EDTA, pyruvate, lactate, glutamine, glucose, bovine serum albumin, and DNAse I. The pH of the injection media is suitably in the range of 7.0-7.7, but as will be understood by the skilled artisan, can be adjusted to be more basic or more acidic depending upon the medium composition, the cell type and/or concentration, and the microenvironment of the recipient injection site.

In another embodiment, other systems can be used for the introduction of one or more SSCs into a recipient male livestock animal. These include injection into the vas deferens and epididymis or manipulations on fetal or juvenile testes, techniques to sever the seminiferous tubules inside the testicular covering, with minimal trauma, which allow injected cells to enter the cut ends of the tubules. Alternatively, neonatal testis (or testes), which are still undergoing development, can be used. SSCs entering the testicular tubule are generally protected from destruction by the immunologically privileged environment of the internal lumen of the tubule. Cells that leak from the tubule are typically destroyed by the immune system of the host since the cells are foreign to the animal.

In some aspects, the disclosed methods can be expanded and incorporated into strategies to generating transgenic livestock animals. The methods of this aspect can be used to provide animals with a single, or many, genetic modification(s) or novel characteristic(s). The present disclosure can also be advantageously applied to livestock animals to imbue these animals with advantageous genetic modification(s) or characteristic(s). The modifications can be implemented into the SSCs using known techniques, such as techniques incorporating, e.g., CRISPR/Cas9, TALEN, and the like.

In yet other aspects, the methods of the disclosure can be incorporated into treatments to maintain or restore fertility. For example, in some embodiments, the donor and recipient livestock animal are the same individual subject. For example, a population of cells comprising SSCs are collected from a livestock animal prior to destruction of the germ cell population and then reintroduced thereafter. This embodiment would preserve the ability of the livestock animal to reproduce following, e.g., radiation or chemical-based therapy. Alternatively, spermatogonial stem cells can be harvested from the livestock animal and kept in culture or frozen. In this aspect of the disclosure, when progeny are desired, the stem cells are transplanted to a recipient testis. A donor livestock animal egg can then be fertilized by spermatozoa developed in the recipient male livestock animal testis. There are no time constraints on this procedure since the stem cells continually undergo self-renewal.

Addressing various aspects described above, general methods of fertilization of eggs, subsequent culture, and implantation for development of progeny are known in the art and will not be discussed in detail herein. By way of a non-limiting example, methods of fertilizing eggs include, but are not limited to, intracytoplasmic sperm injection (ICSI), round spermatid injection (ROSI), and the like. Additionally, by way of a non-limiting example, methods of fertilizing progeny include, but are not limited to, ICSI, ROSI, and the like.

Once an initial fertilization event is achieved and the resulting offspring is fertile, the mammal line (e.g., with a novel genetic modification or characteristic) is established where the genetic characteristics are present in both male and female offspring. Thus, in accordance with embodiments of the disclosed methods, a livestock animal sire can be produced harboring, in its testes only, a biologically functional germ cell that is not endogenous to that individual by repopulating its testicular seminiferous tubules. This (parent) livestock animal can produce progeny. Every cell in the progeny is genetically non-native as compared to the parent livestock animal.

Both the parent livestock animal and its progeny provided by the present disclosure have multiple and varied uses, including, but not limited to, uses in agriculture and biomedicine. An illustrative agricultural use of the present disclosure relates to increasing the breeding potential of a valuable stud animal. In another aspect of the present disclosure, chimeric livestock animals useful in either biomedicine or agriculture are provided. As will be understood by the skilled artisan, when armed with the present application, the present disclosure provides an advantageous complementation to existing transgenic livestock animal techniques.

These aspects of the present disclosure alleviate the present difficulty and expense of embryological transgenic work. In certain embodiments, spermatogonial stem cells can be genetically modified and then transferred to recipient livestock animal testis. The valuable genetic traits present in the resultant germ cells can be passed onto the (transgenic) progeny of the recipient stud. This particular application is important for the genetic engineering of large agricultural animals.

In another aspect, the disclosure provides a kit for maintaining spermatogonial stem cells (SSCs) in a feeder system. The disclosure includes various kit embodiments that comprise a culture system for the maintenance or proliferation of at least one SSC. Although exemplary kits are described below, the contents of other useful kits will be apparent to the skilled artisan in light of the present disclosure and are also encompassed by the disclosure.

In one embodiment, the kit comprises: a culture system comprising a plurality of endothelial feeder cells, a media suitable for SSC cell growth, and instructional material, wherein said instructional material comprises instructions for the use of said kit to maintain the SSCs in the culture system.

In some embodiments, the kit further comprises a container with one or more culture chambers configured to receive the medium containing the endothelial feeder cells and the SSCs. Such containers include, for example, flasks or plates with, e.g., individual wells, that are appropriately configured for tissue culture applications. In some embodiments, the container comprises a gelatin coated culture surface.

Exemplary media and components thereof are described in more detail above. In some embodiments, the media comprises one or more growth factors selected from GDNF, FGF2, SDF-1a, CSF-1, FDGF, NGF, and TGF-β, in any combination. Exemplary concentrations for each growth factor are described in more detail above.

Feeder cells are described in more detail above in the context of the disclosed methods and are encompassed by this aspect. Briefly, the feeder cells can be embryonic endothelial cells. In some embodiments, the feeder cells are yolk sac-derived endothelial cells. The yolk sac-derived cells can be primary cells or can be transformed such that they are immortalized. In some embodiments, the feeder cells are characterized by expression of vascular addressin and/or cytoplasmic tyrosine kinase encoded by a fes (fps/fes) oncogene or homologs thereof. In some embodiments, the feeder cells are murine C166 cells. In some embodiments, the cells are pretreated to prevent proliferations. For example, the cell can be pretreated with a mitosis inactivating agent, such as Mitomycin-C.

In other embodiments, the kit further comprises a mitosis inactivating agent, such as Mitomycin-C, and instructions of use with the other components of the kit, e.g., to pretreat the feeder cells prior to application to prevent proliferation during co-culture.

The kit can also comprise additional elements to permit application of the enhanced population of SSCs produced with the kit in a method to introduce one or more SSCs into a recipient male, as described above. For example, the kit can also comprise an applicator. The term “applicator” refers to any device including, but not limited to, a hypodermic syringe, a pipette, a bronchoscope, a nebulizer, and the like, for administering a composition of the disclosure to a mammal. The particular applicator included in the kit will depend on, e.g., the method and/or the composition used to introduce a population of enriched SSCs to a cell, as well as the target animal.

Additionally, the kit can comprise reagents for appropriate formulation of the SSCs for implantation in a recipient male. Such reagents can include therapeutically acceptable carriers.

As used herein, an “instructional material” includes a publication, a recording, a diagram, or any other medium of expression which can be used to communicate the usefulness of a method and/or composition of the disclosure in a kit for maintaining, proliferating, or administering any composition recited herein. The instructional material of the kit of the disclosure may, for example, be affixed to a container which contains a composition of the disclosure or may be shipped together with a container which contains a composition. Alternatively, the instructional material may be shipped separately from the container with the intention that the instructional material and the compound be used cooperatively by the recipient.

### Additional Definitions

Unless otherwise defined herein, scientific and technical terms used in connection with the disclosure shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. The methods and techniques are generally performed according to conventional methods well-known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol. I, CRC Press (1976); Handbook of Biochemistry: Section A Proteins, Vol. II, CRC Press (1976); Essentials of Glycobiology, Cold Spring Harbor Laboratory Press (1999).

The following terms, unless otherwise indicated, shall be understood to have the meanings provided below.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not necessarily limited to.” However, use of the term “comprise” and grammatical variants thereof does not necessarily require additional elements from what is explicitly described or recited. That is to say, the term “comprise” in some embodiments can be limited to only the described or recited elements similar to the exclusive term “consist”. Words using the singular or plural number also include the plural and singular number, respectively. The word “about” indicates a number within range of minor variation above or below the stated reference number. For example, in some embodiments “about” can refer to a number within a range of 10%, 9%, 8%, 7%, 6%, 5%, 4%, 3%, 2%, or 1% above or below the indicated reference number.

Disclosed are materials, compositions, systems, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are provided for the purpose of illustrating, not limiting, the disclosure.

### Example 1

This Example describes a representative embodiment of a spermatogonial culture protocol, including exemplary reagents, encompassed by the present disclosure. This protocol is provided in the context of porcine SSC culture for purposes of illustration only and is not intended to be limiting unless otherwise specified. Thus, the disclosure should in no way be construed as being limited to the following exemplary protocol, but rather, should be construed to encompass any and all variations that become evident as a result of the teaching provided herein.

### Reagents

Collagenase type 4 Solution (0.25 mg/ml, Worthington, cat. LS004189, dissolved in Hank's Balanced Salt Solution, HBSS)
DNase I Solution (7 mg/ml, Sigma, Cat. DN25, dissolved in HBSS)
Trypsin/EDTA Solution (Gibco, Cat. 25200-114)
Hank's Balanced Salt Solution (HBSS, Gibco, Cat. 14175-145)
30% PERCOLL®Solution—30 ml PERCOLL® (Sigma, Cat.P4937), 1 ml Pen/Strep (Gibco, cat. 15140-122), 1 ml FBS (Gibco Cat. 10437-036), 68 ml Dulbecco's Phosphate Buffered Saline (Gibco, Cat. 14190-144)
0.1% Gelatin (Sigma, Cat. G1890) in Deionized Water—Sterilized
Recombinant Human GDNF (PeproTech, cat. 450-10, stock solution of 20 μg/ml)
Recombinant Human FGF2 (PeproTech, cat. 100-18B, stock solution of 1 μg/ml)
Recombinant Human SDF-1a (PeproTech, cat. 300-28A, stock solution of 10 μg/ml)
Recombinant Human CSF-1 (PeproTech, cat. 300-25, stock solution of 10 μg/ml)
Base Media for Porcine Culture (for preparation of 100 ml which should be stored at 4° C. for no more than 14 days):
   95 ml DMEM F12/Glutamax (Gibco, cat. A1000701)
   1 ml Transferrin Stock Solution (10 mg/ml, Sigma, cat. T1283)
   640 μl Insulin Stock Solution (4 mg/ml, Gibco, cat. 12585-014)
   120 μl Putrescine Stock Solution (100 mM, Sigma, cat. p5780)
   20 μl Na₂SeO₃ Stock Solution (0.3 mM, Sigma, cat. s5261)
   100 μl BME Solution (7 μl BME in 1 ml of DMEM F12, Sigma, cat. m3148), final concentration of 100 mM
   1 ml MEM Vitamins (Gibco, cat. 11120-052)
   1 ml MEM NEAA (Gibco, cat. 11140-050)
   1 ml Hepes (Gibco, cat. 15630-080)
   1 ml Glutamine (100×Stock, Gibco, cat. 25030-081)
   0.5 ml Pen/Strep (100×Stock, Gibco, cat. 15140-122)
   2.4 ml BSA Solution (Gibco, cat. A1000701)
   2 ml StemPro hESC Supplement (Gibco, cat. A1000701)

Preparation of C166 Feeder Cells
C166 feeder cells obtained from ATCC, cat #CRL-2581
Growth Media: DMEM (Sigma, cat. 11965-126), 7% FBS (Gibco, Cat. 10437-036), 1×Penicillin/Streptomycin Solution (Gibco, cat. 15140-122), 1×Glutamine Solution (Gibco, cat. 25030-081), 7 μl/L BME (Sigma, cat. m3148)
Maintain at 37° C. in a humidified incubator with atmosphere of 5% CO₂ in air
Mitotic inactivation at ˜80% confluence by incubation with Growth Media containing Mitomycin C (200 μl/10 ml Sigma, cat. M4287) for 4 hrs
Discard Mitomycin C media and wash with HBSS 3×
Add 1 ml of Trypsin/EDTA solution and incubate at 37° C. for 5 minutes
Add 3 ml of growth media to inactivate trypsin and pass cell suspension through 5 ml serological pipette several times to generate single cell suspension
Pellet cells by centrifugation at 600×g for 7 minutes at 4° C. and resuspend in Growth Media for counting.
Prior to collecting Mitomycin-C treated feeders, prepare gelatin coated culture wells by incubating 0.1% gelatin solution for 15 minutes at room temperature and then wash 3× with HBSS, removing the buffer after the last wash and let the plate air dry
Seed ˜9×10⁴ cells per well of a 24 well plate two days before use as feeders
Feeders are viable for use with germ cell cultures for up to 10 days after plating

Generation of Testicular Tissue Single Cell Suspension (Appropriate for Tissue Size Up to 300 mg)
1. Prepare 10 ml of Collagenase type 4 solution and 5 ml of DNase I solution
2. Combine 10 ml of collagenase type 4 solution with 1 ml of DNase to 1 solution in a GentleMACS C Tube
3. When starting with a whole testis that has an intact tunica albuginea, quickly immerse 1× in 70% ethanol
4. Open the testis with a sterile scalpel blade and excise ˜300 mg or less of parenchyma, being sure to not capture any of the rete testis and tunica albuginea, and place onto a sterile petri dish
5. Using fine forceps, gently tease apart the tissue without fragmenting it and add to GentleMACS tube containing the collagenase/DNase solution.
6. Load the tube into the GentleMACS machine and run cycle code: 37C_m_LPDK_1 (preprogrammed)
7. When cycle is complete, remove the tube and let the tissue settle on ice for 5-7 minutes
8. Remove supernatant and add 10 ml of HBSS to wash tissue, use serological pipet to remove large pieces of tissue that have not been disassociated, then allow disassociated tubules to sediment on ice for 5 minutes and again remove supernatant, repeat 3×
9. Add 10 ml of trypsin/EDTA solution and 1 ml of DNase I solution to the washed and sedimented tissue
10. Load tube into GentleMACS machine and again run cycle code: 37C_m_LPDK_1. When complete, if suspension is viscous or mucus-like, add 1 ml DNase I solution and run cycle 37C_m_LPDK_1 again
11. Add 2 ml of FBS and 1 ml of DNase I solution and gently pipet to create a homogenous single cell suspension which can be checked by placing a small drop on a glass slide and microscopic observation
12. Centrifuge at 600×g for 7 minutes at 4° C. to pellet cells
13. Remove supernatant and if there is a “snotty” layer on top of the pellet, add 2 ml of DNase I solution and pipet gently
14. Add 18 ml of HBSS and pass the suspension through a 70 μm cell strainer

Spermatogonial Enrichment
1. Overlay 5 ml of cell suspension onto 2 ml of 30% PERCOLL® (Cytiva, Marlborough, Mass.) solution in a 15 ml conical tube (should be 4 tubes in total)
2. Centrifuge at 600×g for 8 minutes at 4° C.
3. Add 2 ml of 0.1% gelatin solution per well of a 6-well culture plate and incubate at room temperature for 15 minutes, then wash with HBSS
4. From PERCOLL® tubes, remove supernatant from all 15 ml conical tubes and resuspend each cell pellet in 2 ml of HBSS. Combine all cell pellets into a new 15 ml conical tube and wash each PERCOLL® tube with an additional 1 ml of HBSS and add to the combined conical tube
5. Centrifuge at 600×g for 7 minutes at 4° C. and remove supernatant
6. If pellet has a visible red blood cell layer, resuspend in 2 ml of ACK Lysis Buffer (Gibco, cat. A1049201) and incubate for 3 minutes on ice
7. Add 10 ml of HBSS and resuspend cell pellet
8. Centrifuge at 600×g for 7 minutes at 4° C. and remove supernatant
9. Resuspend pellet in 2 ml of porcine base culture media and centrifuge at 600×g for 7 min at 4° C. and remove supernatant
10. Resuspend pellet in 3-6 ml of base media and add to gelatin coated 6-well culture plates (3 ml per well)
11. Incubate plates overnight at 37° C. in a humidified incubator with an atmosphere of 5% CO₂ in air
12. On the next day, collect non-adherent cells by swirling the plate and removing the media into a 50 ml conical tube. Add another 3 ml of media, repeat swirling and collect media into tube. Effectiveness of the recovery can be monitored by microscopic evaluation of the 6-well plate. An alternative is to add HBSS to the plate and use gentle pipetting to remove non-adherent cells

Setup of Primary Spermatogonial Culture
1. Centrifuge collected cells at 600×g for 7 minutes at 4° C. and remove supernatant
2. Resuspend pellet in base media and count cell number using a hemacytometer
3. Pellet cells by centrifugation at 600×g for 7 minutes at 4° C. and remove supernatant
4. Resuspend pellet in base media containing GDNF (20 ng/ml), FGF2 (1 ng/ml), SDF-1a (10 ng/ml), and CSF-1 (10 ng/ml)
5. Media must be changed every other day and spermatogonia are sub-cultured onto fresh feeders every 5-7 days at a concentration of 1-2×10⁵ cells/well

Base culture media does not last long and should be made fresh every 10-14 days.

### Example 2

This Example describes the in vitro culture and maintenance of porcine spermatogonial stem cells (SSCs). The results demonstrate an unexpectedly high (i.e., exponential) expansion of the SSC population. Furthermore, the cultured SSCs were confirmed to have morphologies and genetic markers characteristic of normal germ cells. Finally, the cultured SSCs were successfully transplanted into NANOS2 knockout sires producing normal sperm, demonstrating the success of the culture technique to maintain and expand SSCs in vitro that are capable of spermatogenesis and production of normal sperm.

Endothelial feeder cells (i.e., C166) feeder cells were prepared and maintained as described in Example 1. Testicular tissue was surgically obtained from a male porcine animal and processed to obtain SSCs as described in Example 1. SSCs were maintained and enriched in culture in base media containing GDNF (20 ng/ml), FGF2 (1 ng/ml), SDF-1a (10 ng/ml), and CSF-1 (10 ng/ml) and the prepared endothelial feeder cells as described in Example 1. A detailed list of the porcine SSC culture ingredients and conditions for various tested factors and feeder systems is set forth in TABLE 1.

TABLE 1
Porcine Culture Conditions

| Media Components | | Concentration | |
|---|---|---|---|
| Base | DMEM/F12 | 1X | |
| Serum Replacement | StemPro | 2% | v/v |
| Growth Factors | | | |
| | GDNF | 20 | ng/ml |
| | FGF2 | 2 | ng/ml |
| | CSF-1 | 10 | ng/ml |
| | SDF-1a | 10 | ng/ml |
| BSA | | 0.5% | v/v |
| Selenium | | 6 × 10(−8)M | |
| Iron-Saturated | | 100 | ug/ml |
| Transferrin | | | |
| Insulin | | 25 | ug/ml |
| Putrescine | | 120 | ug/ml |
| NEAA Mix | | 1X | |
| Vitamin Mix | | 1X | |
| Glutamine | | 2 | mM |
| HEPES | | 10 | mM |
| BME | | 100 | uM |
| Penicillin | | 50 | U/ml |
| Streptomycin | | 50 | U/ml |

| | Maintenance | |
|---|---|---|
| | (>1 Month) | Exponential Expansion |
| Feeders | | |
| STO | N | N |
| PFF | Y | Y but limited |
| C166 | Y | Y |
| Environment | | |
| 35 C./21% O2 | N | N |
| 35 C./10% O2 | N | N |
| 37 C./21% O2 | Y | Y |
| 37 C./10% O2 | ? | ? |

The porcine SSCs cultured and expanded in co-culture with endothelial feeder cells were examined for morphology, gene expression, expansion, and functional spermatogenesis characteristics.

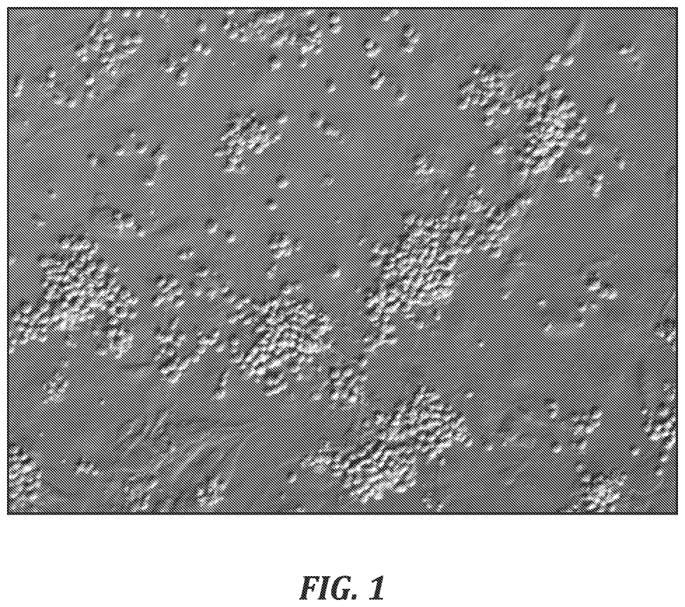

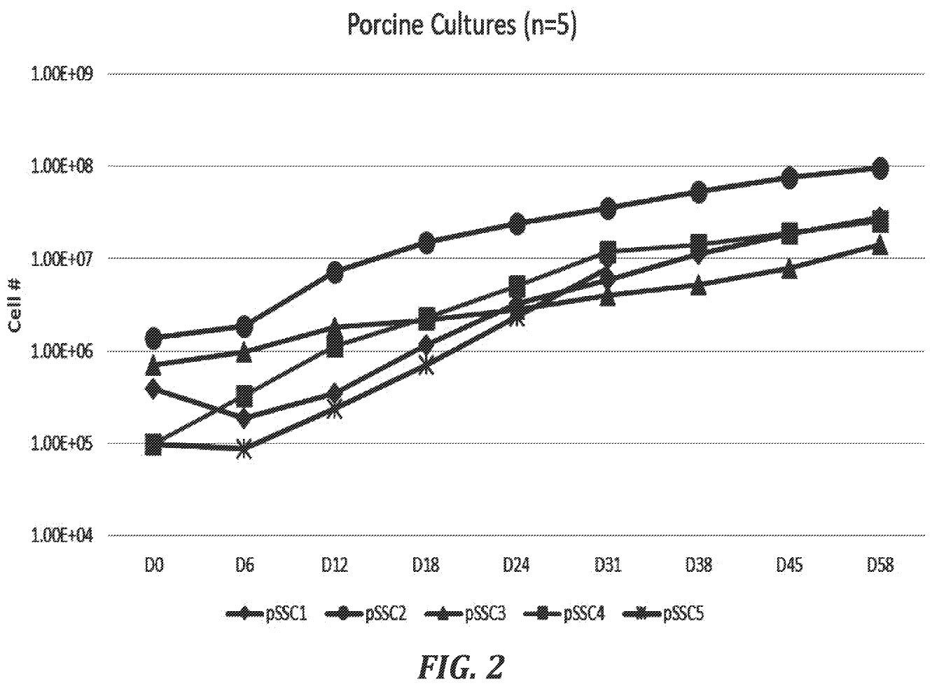

Visual inspection of the SSCs indicated normal morphology after extended in vitro culture. For example, FIG. 1 is a representative image of primary culture of porcine spermatogonia taken after 30 days of in vitro culture, indicating healthy and normal morphology. The cell counts of multiple, independent cultures spermatogonia were monitored for a period of at least two months of maintenance in in vitro. As illustrated in FIG. 2, all cultures exhibited spermatogonia expansion by several orders of magnitude over just 58 days. This demonstrates a remarkable and unexpected capacity for the SSCs to expand in the disclosed culture conditions.

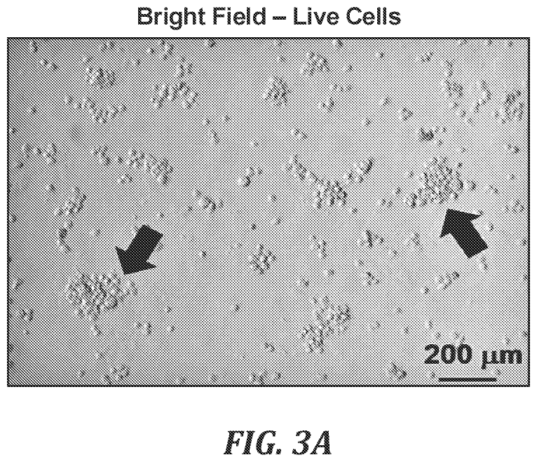

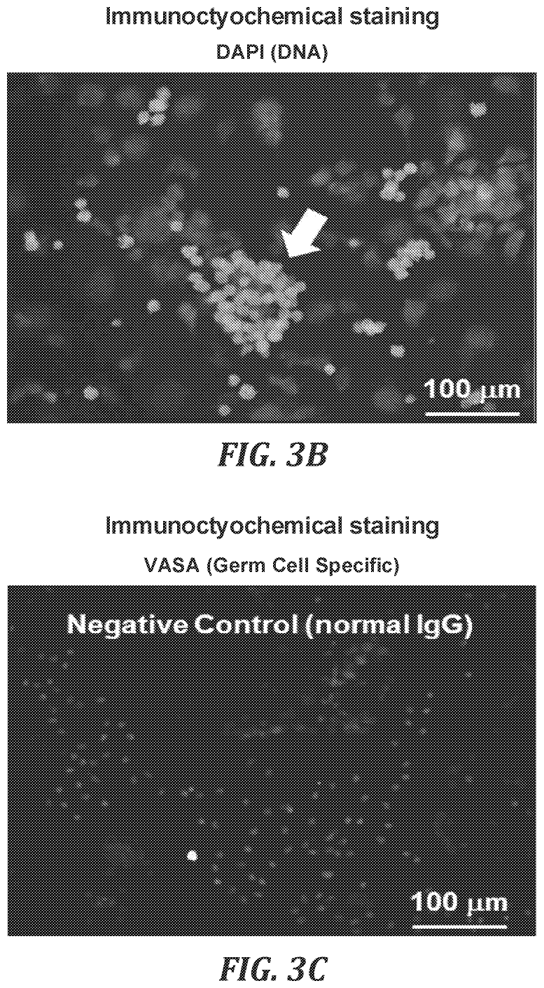

The expanded cells were then assessed for characteristic indicative of the germ status to ensure the primary culture of expanded cell populations did not start to irretrievably differentiate when in culture. FIG. 3A is a representative brightfield image of live spermatogonia cells in culture after 30 days in culture. Immunocytochemical staining of the cells for DNA (using DAPI) and the VASA (a germ-cell specific molecular marker) confirmed that the cultured cells remained viable and retained expression of markers indicative of germ cells. See FIGS. 3AB and 3C. Furthermore, flow cytometric analysis of the primary porcine spermatogonial cultures for a stem cell marker, SOX2, demonstrated that the cultured cells overwhelmingly exhibited molecular characteristics of stem cells. See FIGS. 4A and 4B. This confirms that the cells maintained and expanded in culture retain characteristics of germ cells.

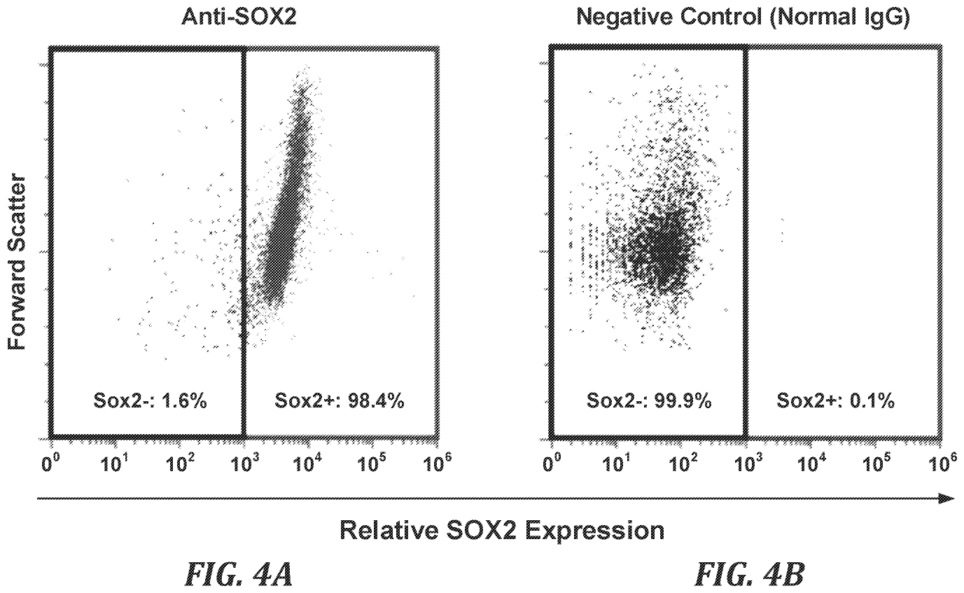

Finally, to determine whether the cultured spermatogonia retain capacity for functional spermatogenesis, the cells cultured for 30 days as described above were implanted into surrogate sire boar that is genetically modified to knockout functional NANOS2 expression. As described in WO 2016/011029, incorporated herein by reference in its entirety, NANOS2 knockout livestock males (e.g., porcine and bovine animals) do not produce endogenous germline cells but retain the somatic tissues, such as Sertoli cells, necessary to support implantation of donor SSCs, leading to functional spermatogenesis of the implanted SSCs and production of viable sperm with the donor genome. Briefly, SSCs cultured and propagated in vitro. After 30 days of culture, approximately 5 million SSCs were injected into the rete testis of each testicle of a homozygous NANOS2 edited (i.e., homozygous NANOS2^{-/-}) recipient male 3.5 year old boar. After SSC transplantation and allowing for spermatogenesis, semen was collected and examined microscopically, confirming the presence of sperm cells. FIG. 5 is a photomicrograph showing an illustrative sperm obtained from a surrogate sire boar receiving SSC transplantation. As illustrated, the morphology appears normal. Additional characteristics, including sperm cell concentration and motility were also assessed (not shown).

These data demonstrate that the disclosed use of endothelial feeder cells contributes to an in vitro culture environment that facilitates remarkable and unprecedented expansion of porcine SSC. The cultured SSCs were exhibited normal characteristics of SSCs, including markers for germ cells. As a practical demonstration of functionality, the cultured cells were successfully implanted in surrogate male sires, leading to spermatogenesis and production of normal sperm. Thus, the disclosed culture platform is highly successful for the expansion of donor SSC cultures to facilitate breeding strategies.

### Example 3

Example 2 describes the successful culture and expansion of porcine SSCs capable of spermatogenesis upon implantation into an appropriate surrogate male sire. To demonstrate that the described culture technique is broadly applicable to SSCs of other livestock animals, the co-culture strategy was repeated for bovine SSCs.

The general methodology for feeder cells preparation, maintenance, and co-culture with bovine SSCs is described in Example 1, with certain modifications. Bovine spermatogonia were isolated from bovine testicular parenchyma according to a methodology described in Oatley, Melissa J., et al. “Conditions for long-term culture of cattle undifferentiated spermatogonia.” Biology of Reproduction 95.1 (2016): 14-1, incorporated herein by reference in its entirety. The disclosed base media for co-culture of bovine SSCs with the endothelial (i.e., C166) feeder cells, was modified to include the following growth factors GDNF, FGF2, CSF-1, SDF-1a, PDGF, NGF, and TGF-beta. Culture of the SSCs was generally maintained at about 35° C. and 10% O₂ tension. A detailed list of the bovine SSC culture ingredients and conditions for various tested factors and feeder systems is set forth in TABLE 2.

TABLE 2
Bovine SSC Culture Conditions

| Media Components | | Concentration | |
|---|---|---|---|
| Base | MEMa | 1X | |
| Serum Replacement | StemPro | 2% | v/v |
| BSA | | 0.5% | v/v |
| Selenium | | 6 × 10⁻⁸M | |
| Iron-Saturated Transferrin | | 100 | ug/ml |
| Insulin | | 25 | ug/ml |
| Putrescine | | 120 | ug/ml |
| NEAA Mix | | 1X | |
| Vitamin Mix | | 1X | |
| Glutamine | | 2 | mM |
| HEPES | | 10 | mM |
| BME | | 100 | uM |
| Penicillin | | 50 | U/ml |
| Streptomycin | | 50 | U/ml |
| Growth Factors | | | |
| | GDNF | 20 | ng/ml |
| | FGF2 | 2 | ng/ml |
| | CSF-1 | 10 | ng/ml |
| | SDF-1a | 10 | ng/ml |
| | PDGF | 10 | ng/ml |
| | NGF | 10 | ng/ml |
| | TGF-beta | 10 | ng/ml |

| | Maintenance 1 | Maintenance 3 |
|---|---|---|
| | Month | Months |
| Feeders | | |
| STO | N | N |
| BFF | Y | Y |
| C166 | Y | Y |
| C166 + BFF | Y | N |
| Feeder-Free (BFF Conditioned | Y | N |
| Media) | | |
| C166 + BFF Conditioned Media | Y | Y |
| Environment | | |
| 35° C./21% O₂ | N | N |
| 35° C./10% O₂ | Y | Y |
| 37° C./21% O₂ | N | N |
| 37° C./10% O₂ | Y | N |

The bovine SSCs cultured and expanded in co-culture with endothelial feeder cells were examined for morphology and expansion characteristics.

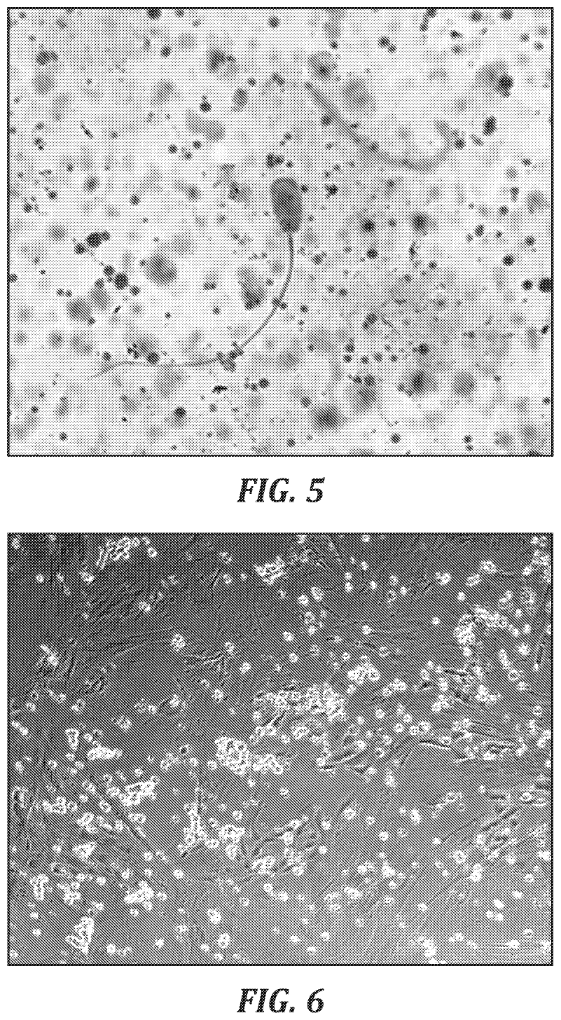

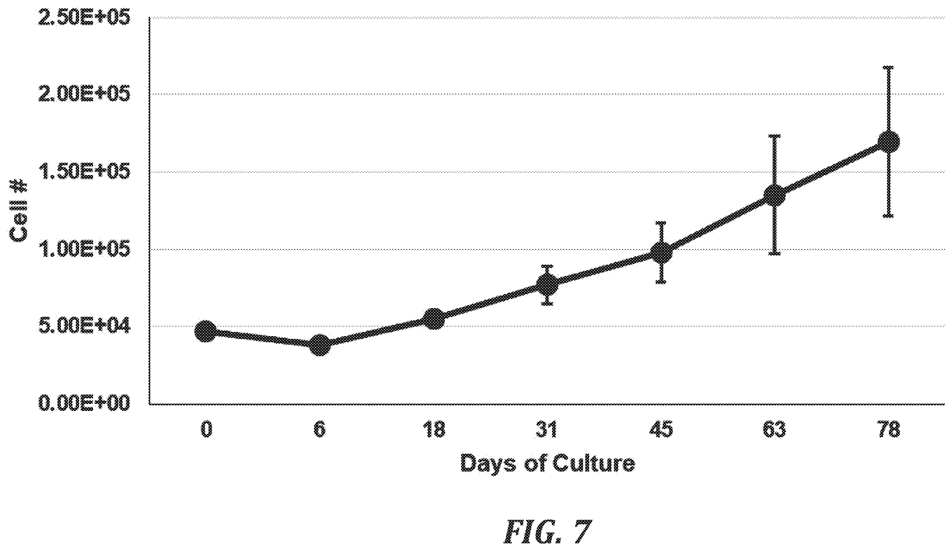

Visual inspection of the bovine SSCs indicated normal morphology after extended in vitro culture in the described conditions. For example, FIG. 6 is a representative image of primary culture of bovine spermatogonia taken after 30 days of in vitro culture, indicating healthy and normal morphology. The cell count of spermatogonia in culture was monitored for a period of about 80 days of maintenance in vitro. As illustrated in FIG. 7, the numbers of spermatogonia expanded several orders of magnitude over a 78 day period. Similar to the culture of porcine SSCs described in Example 2, these data demonstrate a remarkable and surprising capacity for the disclosed culture conditions to support maintenance and expansion of SSCs in vitro.

Additional characterization will be performed to confirm the germ status of the cultured cells and to confirm their capacity to colonize testes material in a surrogate male's testes and thereafter undergo spermatogenesis to produce viable sperm in the surrogate male. Such assays are described in Example 2 in the context of porcine SSCs. For example, the cultured bovine SSCs will be subjected to immunocytochemical staining for germ cell-specific markers, including VASA and SOX2. The status of the markers can be assessed using fluorescence microscopy and/or flow cytometry. Based on the positive results with porcine SSCs, described in Example 2, and the observed normal morphology and capacity for expansion of the bovine SSCs, the SSCs are expected to express markers characteristic of germ/stem cells.

After about 1-3 months of culture and expansion, about 1-10 million cultured bovine SSCs will be injected into each testis of homozygous NANOS2 edited (i.e., homozygous NANOS2^{-/-}) recipient male bovine animal. Typical recipient NANOS2 edited male bovines are prepubertal, e.g., less than 10 months old, such as 1-4 months old. Once the recipient male reaches puberty and sufficient time for spermatogenesis has passed, semen will be collected and examined for presence of sperm cells. If present, the sperm cells will be observed and characterized for concentration, motility, and morphology. Genetic profiling will be conducted to confirm the sperm are derived from the cultured SSCs and not the surrogate male recipient.

The present data indicate that the successful in vitro culturing platform incorporating endothelial feeder cells, as described above for porcine, is also applicable to bovine animals. As with the observed porcine results, the culturing platform resulted in remarkable and unprecedented (i.e., exponential) expansion of SSCs, which maintained their observable morphology.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of enriching spermatogonial stem cells (SSCs) from a population of testis-derived cells containing at least one SSC, wherein the population of cells is derived from the testis of a livestock animal, the method comprising:
contacting said population of testis-derived cells to a culture media comprising endothelial feeder cells, or parts thereof, and
maintaining culture conditions suitable for SSC cell maintenance and enrichment.

2. The method of claim 1, wherein the endothelial feeder cells are maintained in a layer in or on a gelatin coated culture surface.

3. The method of claim 1 or claim 2, wherein the feeder cells are embryonic endothelial cells.

4. The method of one of claim 1 to claim 3, wherein the feeder cells are yolk sac-derived endothelial cells.

5. The method of one of claim 1 to claim 4, wherein the feeder cells are characterized by expression of vascular addressin.

6. The method of one of claim 1 to claim 5, wherein the feeder cells are characterized by expression of cytoplasmic tyrosine kinase encoded by a fes (fps/fes) oncogene or homologs thereof.

7. The method of one of claim 1 to claim 6, wherein the feeder cells are murine C166 cells.

8. The method of one of claim 1 to claim 7, wherein the feeder cells are pre-treated with a mitosis inactivating agent, such as Mitomycin-C.

9. The method of one of claim 1 to claim 8, wherein the culture media further comprises one or more growth factors selected from glial cell-derived neurotrophic factor (GDNF), fibroblast growth factor 2 (FGF2), stromal cell-derived factor 1 (SDF-1a), colony stimulating factor 1 (CSF-1), fibroblast-derived growth factor (FDGF), nerve growth factor (NGF), and transforming growth factor beta (TGF-β), in any combination.

10. The method of claim 9, wherein the culture media comprises GDNF, FGF2, SDF-1a, and CSF-1.

11. The method of claim 10, wherein the culture media further comprises DGF, NGF, and TGF-β.

12. The method of one of claim 1 to claim 11, further comprising isolating enriched SSCs from the culture media.

13. The method of one of claim 1 to claim 12, wherein the SSCs are porcine SSCs.

14. The method of one of claim 1 to claim 12, wherein the SSCs are bovine SSCs

15. The method of claim 1, wherein the SSCs are porcine SSCs, wherein the endothelial feeder cells comprise murine C166 cells, and wherein the culture media further comprises GDNF, FGF2, SDF-1a, and CSF-1.

16. The method of claim 1, wherein the SSCs are bovine SSCs, wherein the endothelial feeder cells comprise murine C166 cells, and wherein the culture media further comprises GDNF, FGF2, SDF-1a, CSF-1, DGF, NGF, and TGF-0.

17. A method of enriching spermatogonial stem cells (SSCs) from a population of testis-derived cells containing at least one SSC, wherein the population of cells is derived from the testis of a livestock animal, the method comprising:
contacting the population of testis-derived cells to a culture media that has been preconditioned with a feeder cell line of endothelial cells, or parts thereof, and
maintaining culture conditions suitable for SSC cell maintenance and enrichment.

18. The method of claim 17, wherein the culture media is preconditioned by contacting the media with the endothelial feeder cells which are maintained in a layer in or on a gelatin coated culture surface.

19. The method of claim 17 or claim 18, wherein the feeder cells are embryonic endothelial cells.

20. The method of claim 17 or claim 19, wherein the feeder cells are yolk sac-derived endothelial cells.

21. The method of one of claim 17 to claim 19, wherein the feeder cells are characterized by expression of vascular addressin.

22. The method of one of claim 17 to claim 21, wherein the feeder cells are characterized by expression of cytoplasmic tyrosine kinase encoded by a fes (fps/fes) oncogene or homologs thereof.

23. The method of one of claim 17 to claim 22, wherein the feeder cells are murine C166 cells.

24. The method of one of claim 17 to claim 23, wherein the feeder cells are treated with a mitosis inactivating agent, such as Mitomycin-C, prior to or during contact with the culture media.

25. The method of one of claim 17 to claim 24, wherein the culture media further comprises one or more growth factors selected from GDNF, FGF2, SDF-1a, CSF-1, FDGF, NGF, and TGF-β, in any combination.

26. The method of claim 25, wherein the culture media comprises GDNF, FGF2, SDF-1a, and CSF-1.

27. The method of claim 26, wherein the culture media further comprises FDGF, NGF, and TGF-β.

28. The method of one of claim 17 to claim 24, further comprising preconditioning the culture media with the feeder cell line of endothelial cells, followed by removing the feeder cell line of endothelial cells or substantially all of the feeder cell line of endothelial cells from the preconditioned culture media prior to contacting preconditioned culture media to the population of testis-derived cells.

29. The method of one of claim 17 to claim 28, further comprising isolating enriched SSCs from the preconditioned culture media.

30. The method of one of claim 17 to claim 29, wherein the SSCs are porcine SSCs.

31. The method of one of claim 17 to claim 29, wherein the SSCs are bovine SSCs.

32. The method of claim 17, wherein the SSCs are porcine SSCs, wherein the endothelial cells comprise murine C166 cells, and wherein the culture media further comprises GDNF, FGF2, SDF-1a, and CSF-1.

33. The method of claim 17, wherein the SSCs are bovine SSCs, wherein the endothelial cells comprise murine C166 cells, and wherein the culture media further comprises GDNF, FGF2, SDF-1a, CSF-1, DGF, NGF, and TGF-β.

34. A population of enriched SSC produced by the method of one of claim 1 to claim 33.

35. A method of generating at least one livestock animal progeny, said method comprising:
a) administering a population of enriched SSCs as recited in claim 34 to a testis of a recipient male livestock animal;
b) allowing said enriched SSCs to generate a colony of spermatogenesis in said recipient male livestock animal; and
c) mating said recipient male livestock animal with a female male livestock mammal of the same species as said recipient male livestock animal.

36. The method of claim 35, wherein the population of enriched SSCs is administered to the rete testis of said recipient male livestock animal.

37. The method of claim 35, wherein the population of enriched SSCs is administered to the lumen of a seminiferous tubule of said recipient male livestock animal.

38. The method of one of claim 35 to claim 37, wherein the SSCs are porcine cells and the recipient livestock animal and the female livestock animal are porcine animals.

39. The method of one of claim 38 to claim 37, wherein the SSCs are bovine cells and the recipient male livestock animal and the female livestock animal are bovine animals.

40. The method of one of claim 35 to claim 39, wherein said recipient male livestock animal is genetically modified to reduce or eliminate NANOS2 function.

41. A kit for maintaining spermatogonial stem cells (SSCs) in a feeder system, said kit comprising:
a) a culture system comprising a plurality of endothelial feeder cells and a medium suitable for SSC cell growth;
b) an applicator, and
c) instructional material, wherein said instructional material comprises instructions for the use of said kit to maintain the SSCs in the culture system.

42. The kit of claim 41, further comprising a container with one or more culture chambers configured to receive the medium containing the endothelial feeder cells and the SSCs

43. The kit of claim 42, wherein the container comprises a gelatin coated culture surface.

44. The kit of claim 41, wherein the feeder cells are embryonic endothelial cells.

45. The kit of claim 41, wherein the feeder cells are yolk sac-derived endothelial cells.

46. The kit of claim 41, wherein the feeder cells are characterized by expression of vascular addressin and/or cytoplasmic tyrosine kinase encoded by a fes (fps/fes) oncogene or homologs thereof.

47. The kit of one of claim 41 to claim 46, wherein the feeder cells are murine C166 cells.

48. The kit of one of claim 41 to claim 47, wherein the feeder cells are pretreated with a mitosis inactivating agent, such as Mitomycin-C.

49. The kit of one of claim 41 to claim 47, further comprising a mitotic inactivating agent, such as Mitomycin-C.

50. The kit of one of claim 41 to claim 49, wherein the culture media comprises one or more growth factors selected from GDNF, FGF2, SDF-1a, CSF-1, FDGF, NGF, and TGF-β, in any combination.
